(19) United States
(12) Patent Application Publication
(10) Pub. No.: US 2026/0358392 A1
(43) Pub. Date: Nov. 12, 2026

(54) INFORMATION PROCESSING DEVICE, NON-TRANSITORY COMPUTER-READABLE STORAGE MEDIUM, AND INFORMATION PROCESSING METHOD

(71) Applicant: Mitsubishi Electric Corporation, TOKYO (JP)

(72) Inventors: Koshiro OKUMOTO, Tokyo (JP); Shingo MINE, Tokyo (JP)

(73) Assignee: Mitsubishi Electric Corporation, TOKYO (JP)

(21) Appl. No.: 19/716,459

(22) Filed: Jun. 23, 2026

Related U.S. Application Data
(63) Continuation of application No. PCT/JP2024/008538, filed on Mar. 6, 2024.

(51) Int. Cl. G01R 23/16; G06F 17/16

(52) U.S. Cl. CPC G01R 23/16; G06F 17/16

(57) ABSTRACT

A signal processing device includes a signal acquiring unit configured to acquire a time-series signal indicating a plurality of waveforms; a signal shaping unit configured to identify a single waveform from the time-series signal; and a frequency analysis unit configured to calculate a power parameter that is a parameter indicating power of the single waveform.

### CROSS REFERNCE TO RELATED APPLICATION

This application is a continuation application of International Application No. PCT/JP2024/008538 having an international filing date of Mar. 6, 2024, which is hereby expressly incorporated by reference into the present application.

### BACKGROUND OF THE INVENTION

### 1. Field of the Invention

The present invention relates to an information processing device, a non-transitory computer-readable storage medium, and an information processing method.

### 2. Description of the Related Art

In recent years, techniques have been developed to analyze signals, such as pulse waves, that contain components repeatedly fluctuating up and down, whose waveforms are different from each other.

The pulse-wave analysis device described in Patent Document 1 detects the local minimum values and the local maximum values of a pulse wave, divides waveform values into units each of which corresponds to a single beat, rapidly regenerates the waveform values beat-by-beat multiple times, and thus obtains the spectrum of the pulse wave.

Patent Document 1: Japanese Patent Application Publication No. 7-148126

### SUMMARY OF THE INVENTION

A pulse wave is influenced by hemodynamic biomechanics, including cardiac contractility, aortic valve closure, and systemic vascular resistance. It is known that, in a pulse wave signal, pulse wave period, pulse wave power, and pulse wave shape reflect not only normal or abnormal pulse conditions such as arrhythmia or atrial fibrillation, but also emotional states as a result of autonomic nervous system function. For example, autonomic nervous system function can be evaluated by analyzing pulse wave power.

However, although calculating a spectrum of a pulse wave by pulse wave signal processing, conventional techniques neither disclose nor suggest extraction and utilization of pulse wave power. Therefore, the conventional techniques have a problem in that they cannot take into account fluctuation components within a single waveform due to hemodynamic biomechanics, and thus cannot perform accurate analysis.

To cope with this, an object of one or more embodiments of the present disclosure is to enable calculation of a power parameter for each wave from a time-series signal.

An information processing device according to one aspect of the present disclosure includes: a processor to execute a program; and a memory to store the program which, when executed by the processor, performs processes of, acquiring a time-series signal indicating a plurality of waveforms; identifying a single waveform from the time-series signal; and calculating a power parameter that is a parameter indicating power of the single waveform, a period parameter that is a parameter indicating a period of the single waveform, and a waveform shape parameter that is a spectrum calculated by normalizing a frequency spectrum by using a square root of sum of squares, the period parameter being calculated by a square root of sum of squares of the frequency spectrum.

According to one or more embodiments of the present disclosure, it is possible to calculate a power parameter for each wave from a time-series signal.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a diagram schematically illustrating a configuration of a signal processing device according to a first embodiment;

FIG. 2 is a schematic diagram illustrating an example of a waveform indicated by a time series signal;

FIG. 3 is a block diagram schematically illustrating a configuration of a computer;

FIG. 4 is a flowchart of frequency analysis processing;

FIG. 5 is a diagram schematically illustrating decomposition of a waveform shape and power;

FIG. 6 is a block diagram schematically illustrating a configuration of an emotion recognition system according to second and third embodiments;

FIG. 7 is a block diagram schematically illustrating a configuration of a learning device 200 according to the second embodiment;

FIG. 8 is a block diagram schematically illustrating a configuration of an inference device according to the second and third embodiments;

FIG. 9 is a block diagram schematically illustrating a configuration of a learning device according to the third embodiment; and

FIG. 10 is a block diagram schematically illustrating a configuration of a signal reconstruction device according to a fourth embodiment.

### DETAILED DESCRIPTION OF THE INVENTION

### First Embodiment

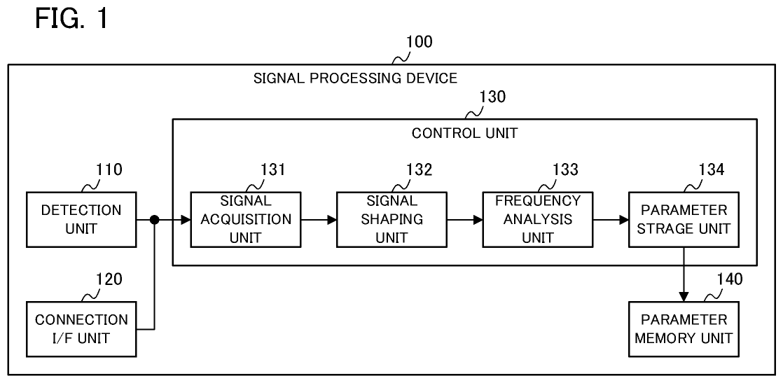

FIG. 1 is a block diagram schematically illustrating a configuration of a signal processing device 100 that is an information processing device according to a first embodiment.

The signal processing device 100 includes a detection unit 110, a connection interface (I/F) unit 120, a control unit 130, and a parameter storage unit 140.

FIG. 1 illustrates that the signal processing device 100 is equipped with both the detection unit 110 and the connection I/F unit 120; however, the signal processing device 100 may include at least one of them.

The detection unit 110 detects a physical quantity related to a subject, and provides the control unit 130 with a time series signal that is a signal having a component that repeatedly fluctuates up and down, such as a pulse wave, corresponding to the detected physical quantity.

The connection I/F unit 120 is a functional unit capable of coupling an external storage device (not illustrated in the drawing), and of transferring data from or to the external storage device. The external storage device is an optical disc, a flash memory storage device, or the like. It is assumed here that the external storage device stores the above-mentioned time series signal. The connection I/F unit 120 reads out the time series signal stored in the external storage device, and provides the time series signal to the control unit 130.

The control unit 130 controls processing in the signal processing device 100.

The control unit 130 includes a signal acquisition unit 131, a signal shaping unit 132, a frequency analysis unit 133, and a parameter storage unit 134.

The signal acquisition unit 131 acquires the time series signal from the detection unit 110 or the external storage device via the connection I/F unit 120. The acquired time series signal is provided to the signal shaping unit 132. The time series signal is a signal indicating a plurality of waveforms.

The signal shaping unit 132 is an identification unit that identifies a single waveform from the time-series signal from the signal acquisition unit 131 and sequentially generates a single waveform signal corresponding to the identified single waveform. The generated single waveform signal is provided to the frequency analysis unit 133.

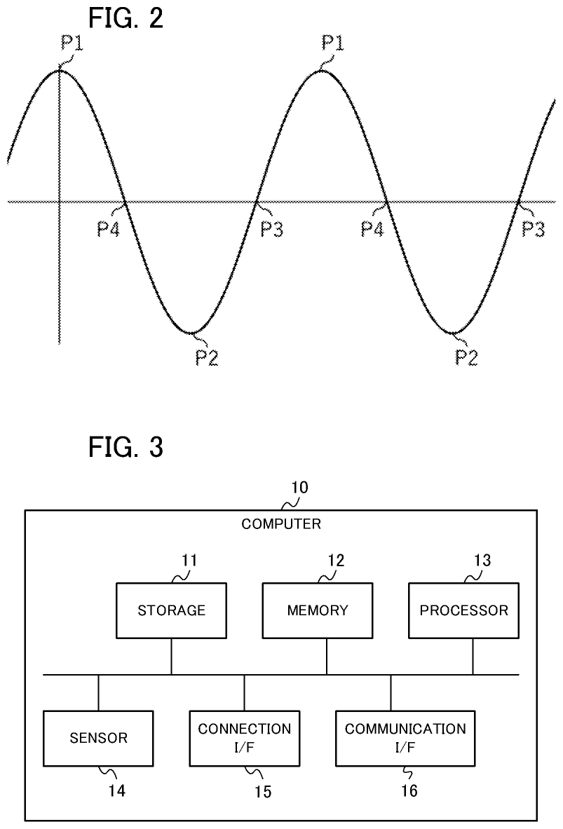

For example, as illustrated in FIG. 2, the signal shaping unit 132 may sequentially capture each waveform by detecting a zero-crossing position P3 in the rising of the waveform represented by the time-series signal.

The position at which a single waveform is captured does not have to be limited to the zero-crossing position P3 in the rising; the position can also be a peak P1, a bottom P2, or a zero-crossing position P4 in the falling of the waveform. Alternatively, a single waveform may be defined and extracted by other methods.

If any characteristic point such as the zero-crossing position P3 cannot be detected due to long-term fluctuation components contained in the time-series signal other than the periodicity, the signal shaping unit 132 may perform processing after removing the long-term fluctuation components by calculating differences by means of polynomial fitting or by calculating differences by means of a moving average.

The frequency analysis unit 133 is a calculation unit that performs frequency analysis on the single waveform signal from the signal shaping unit 132 to parameterize the single waveform represented by the single waveform signal into three elements: a period, power, and a waveform shape. Accordingly, the frequency analysis unit 133 acquires a period parameter, a power parameter, and a waveform shape parameter corresponding to the single waveform. The acquired period parameter, power parameter, and waveform shape parameter are provided to the parameter storage unit 134.

For example, the frequency analysis unit 133 calculates the period of the single waveform or the frequency of the fundamental wave of the single waveform, performs frequency analysis processing, thereby calculating the power parameter. This frequency analysis processing includes a complex Fourier series expansion. Specifically, the frequency analysis unit 133 calculates a frequency spectrum by a complex Fourier series expansion, calculates the square root of sum of squares by normalizing the frequency spectrum by the square root of sum of squares, and then can set the resulting square root of sum of squares as the power parameter.

The parameter storage unit 134 stores the period parameter, the power parameter, and the waveform shape parameter from the frequency analysis unit 133, in a parameter memory unit 140.

The parameter memory unit 140 stores the period parameter, the power parameter, and the waveform shape parameter.

The signal processing device 100 described above can be implemented by a mobile terminal, such as a smartwatch, or a personal computer (PC), corresponding to a computer 10 illustrated in FIG. 3.

The computer 10 includes a storage 11 such as a hard disk drive (HDD), a solid state drive (SSD), or a non-volatile memory; a non-volatile memory 12; a processor 13 such as a central processing unit (CPU); a sensor 14; and a connection I/F 15.

The computer 10 may include a communication I/F 16 such as a network interface card (NIC) for communication with a network.

For example, the parameter memory unit 140 can be implemented by the storage 11 or the memory 12.

The control unit 130 can be implemented by the processor 13 that loads a program stored in the storage 11 into the memory 12 and executes the program.

The detection unit 110 can be implemented by the sensor 14.

The connection I/F unit 120 can be implemented by the connection I/F 15 compliant with Universal Serial Bus (USB) or the like.

As described above, at least one of the sensor 14 and the connection I/F 15 may be provided.

The computer 10 may also include a display interface, which is not illustrated in the drawing, for displaying a processing result depending on the application. Thus, the signal processing device 100 can include a display unit (not illustrated) for displaying a processing result.

The program may be downloaded to the storage 11 from a recording medium not illustrated via a reader/writer not illustrated, or from a network via a communication I/F not illustrated, and then loaded into the memory 12, and executed by the processor 13. Alternatively, the program may be loaded directly into the memory 12 from the recording medium via the reader/writer, or from the network via the communication I/F, and then executed by the processor 13.

In other words, the program may be provided by a computer program product such as a recording medium.

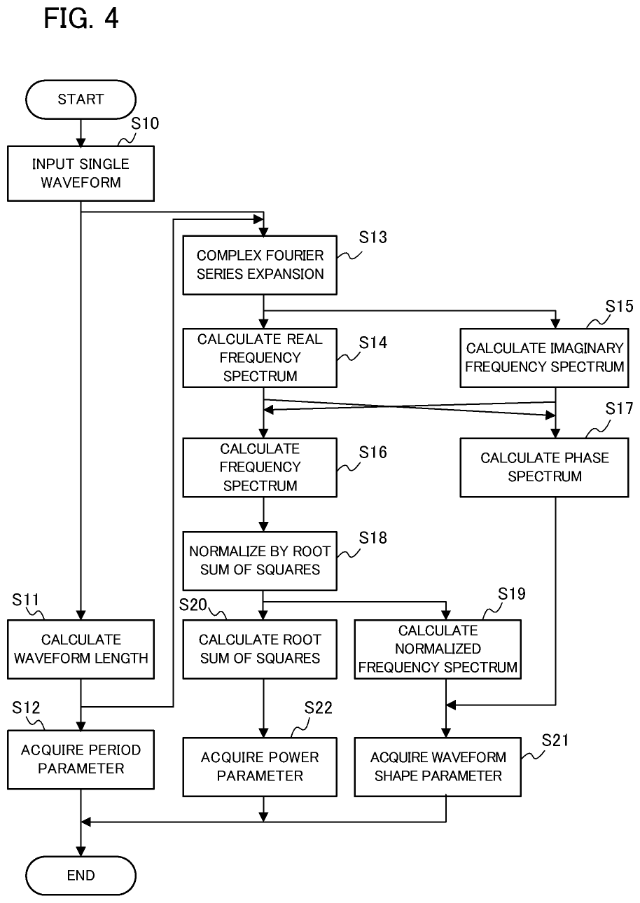

FIG. 4 is a flowchart of frequency analysis processing by the frequency analysis unit 133.

First, the frequency analysis unit 133 receives an input of a single waveform signal from the signal shaping unit 132 (S10), and extracts a single waveform represented by the single waveform signal.

Next, the frequency analysis unit 133 calculates the waveform length from the single waveform extracted in Step S10 (S11).

Then, the frequency analysis unit 133 acquires a period parameter from the calculated waveform length (S12). For example, the frequency analysis unit 133 may calculate the fundamental frequency by taking the reciprocal of the waveform length calculated in Step S11, and set the fundamental frequency as the period parameter. Alternatively, the waveform length itself can be used as the period parameter.

Next, the frequency analysis unit 133 performs a frequency transformation based on a complex Fourier series expansion on the single waveform extracted in Step S10 (S13).

Then, the frequency analysis unit 133 calculates a real Fourier spectrum (S14).

The frequency analysis unit 133 also calculates an imaginary Fourier spectrum (S15).

For example, in the complex Fourier series expansion, the frequency analysis unit 133 calculates n frequency components in total from the fundamental frequency up to n times the fundamental frequency (n is an integer of 2 or more).

Next, the frequency analysis unit 133 calculates the frequency spectrum from the square root of sum of squares of each frequency component of the real Fourier spectrum calculated in Step S14 and the imaginary Fourier spectrum calculated in Step S15 (S16).

The frequency analysis unit 133 also calculates a phase spectrum from the arctangent of each frequency component of the real Fourier spectrum calculated in Step S14 and the imaginary Fourier spectrum calculated in Step S15 (S17).

For example, if the calculation is performed up to five times the frequency, a frequency spectrum and a phase spectrum composed of five frequency components are obtained.

Here, in Step S17, the frequency analysis unit 133 calculates the phase spectrum as the initial phase value of each frequency spectrum, within the range of −π to π. However, although −π and π represent the same phase, they are numerically at the farthest positions from each other, which may limit their use as feature quantities. Accordingly, it is desirable that the frequency analysis unit 133 take the cosine of the phase to calculate the real phase spectrum, and take the sine of the phase to calculate the imaginary phase spectrum.

Next, the frequency analysis unit 133 normalizes the frequency spectrum obtained in Step S16 in the frequency direction by using the square root of sum of squares (S18).

Accordingly, the frequency analysis unit 133 calculates a normalized frequency spectrum (S19).

The frequency analysis unit 133 also calculates the square root of sum of squares of the frequency spectrum normalized in Step S18 (S20).

Then, the frequency analysis unit 133 acquires, as waveform shape parameters, the normalized frequency spectrum calculated in Step S19 and the phase spectrum calculated in Step S17 (S21). When the real phase spectrum and the imaginary phase spectrum are calculated in Step S17, it is desirable that these should be acquired as waveform shape parameters.

The frequency analysis unit 133 also acquires the square root of sum of squares calculated in Step S20 as the power parameter (S22).

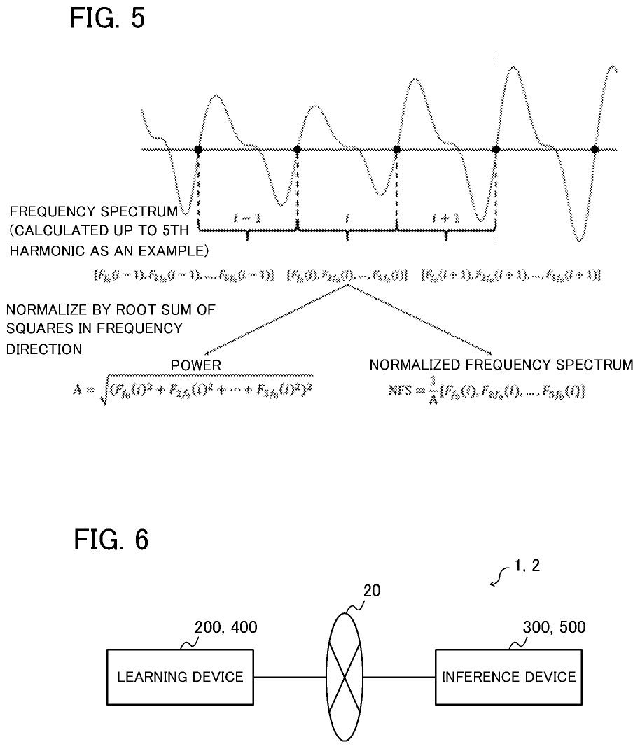

Here, FIG. 5 schematically illustrates decomposition of a waveform shape and power.

Through the above processing, the frequency analysis unit 133 achieves parameterization of the period, the waveform shape, and the power.

As a result, by calculating the power of the waveform by using the square root of sum of squares of the spectrum from the fundamental wave to the harmonics, the frequency analysis unit 133 can achieve more appropriate calculation of the power by taking into account the spectrum from the fundamental wave to the harmonics, than by using the amplitude value of the waveform, in other words, the peak value, as the power. The frequency analysis unit 133 can also calculate the waveform shape independently of the overall amplitude of the signal.

As described above, in the first embodiment, since the frequency analysis unit 133 processes the time-series signal for each waveform and the parameter storage unit 113 continuously stores the processing results, it is possible to acquire temporal variations of the period parameter, the waveform shape parameter, and the power parameter of the time-series signal.

### Second Embodiment

FIG. 6 is a block diagram schematically illustrating a configuration of an emotion recognition system 1 as an information processing system according to a second embodiment.

The emotion recognition system 1 includes a learning device 200 as an information processing device, and an inference device 300 as an information processing device.

Here, the learning device 200 and the inference device 300 are capable of transmitting and receiving data via a network 20, such as the Internet.

The learning device 200 performs processing in a learning phase, which is a phase for training a learning model.

The inference device 300 acquires a trained learning model from the learning device 200, and performs processing in an inference phase, which is a phase where the trained learning model is used to detect emotions from a pulse wave signal that is a time-series signal. The pulse wave signal is a signal representing a pulse wave detected from a person.

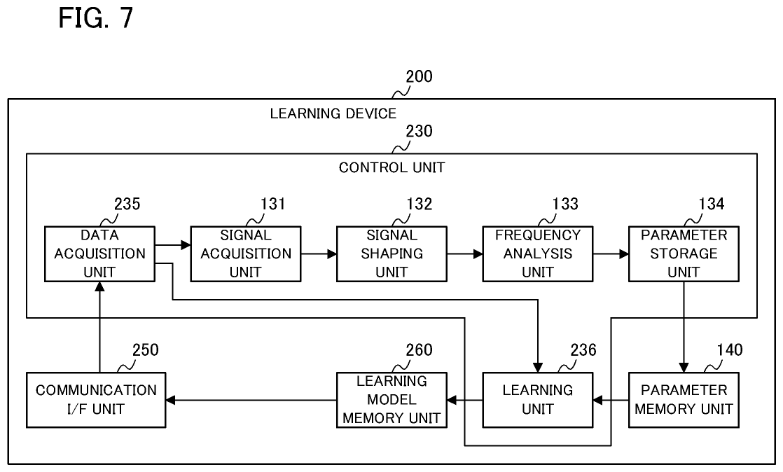

FIG. 7 is a block diagram schematically illustrating a configuration of the learning device 200 according to the second embodiment.

The learning device 200 includes a control unit 230, a parameter memory unit 140, a communication I/F unit 250, and a learning-model memory unit 260.

The parameter memory unit 140 of the learning device 200 according to the second embodiment is similar to the parameter memory unit 140 of the signal processing device 100 according to the first embodiment.

The communication I/F unit 250 performs communication via the network 20.

The control unit 230 controls processing in the learning device 200.

The control unit 230 includes a signal acquisition unit 131, a signal shaping unit 132, a frequency analysis unit 133, a parameter storage unit 134, a data acquisition unit 235, and a learning unit 236.

The signal acquisition unit 131, the signal shaping unit 132, the frequency analysis unit 133, and the parameter storage unit 134 of the control unit 230 in the second embodiment are similar to the signal acquisition unit 131, the signal shaping unit 132, the frequency analysis unit 133, and the parameter storage unit 134 of the control unit 130 in the first embodiment.

However, the signal acquisition unit 131 acquires a time series signal from the data acquisition unit 235, as described later.

The data acquisition unit 235 acquires training data that includes a pulse wave signal as a time-series signal and a label indicating the emotion of a subject from which the pulse wave signal has been acquired. Here, the label is assumed to indicate the emotion of the subject for each predetermined period of the pulse wave signal. Here, the subject from which the pulse wave signal has been acquired is assumed to be a human, and this person is also referred to as a learning subject.

The data acquisition unit 235 may acquire the training data via the communication I/F unit 250 from another device not illustrated, or may acquire the training data by reading the training data from a memory unit not illustrated. The pulse wave signal included in the acquired training data is provided to the signal acquisition unit 131, and the label is provided to the learning unit 236.

The learning unit 236 trains a learning model for inferring the emotion of the subject from which the pulse wave signal has been acquired, based on the pulse wave signal, by using the period parameter, the power parameter, and the waveform shape parameter for each predetermined period stored in the parameter storage unit 140, and the label corresponding to each period. Here, the power parameter, the period parameter, and the waveform shape parameter used for training are also referred to as a training power parameter, a training period parameter, and a training waveform shape parameter, respectively.

For example, the learning unit 236 generates a learning model for inferring the emotion of a person by using at least the power parameter as a training example for learning the emotion of the person at the time when a single waveform corresponding to the power parameter is acquired. Here, a single waveform identified from a time-series signal used for training is also referred to as a training single waveform.

As a machine learning method used by the learning unit 236, a neural network or a support vector machine may be exemplarily selected; however, other machine learning methods may also be employed. The parameter used for training may include at least one of the period parameter, the power parameter, and the waveform shape parameter. Here, it is assumed that the power parameter is used at least.

The learning-model memory unit 260 stores the learning model trained by the learning unit 236.

The communication I/F unit 250 transmits the learning model stored in the learning-model memory unit 260 to the inference device 300.

The learning device 200 described above can be implemented by the computer 10 illustrated in FIG. 3.

For example, the parameter memory unit 140 and the learning-model memory unit 260 can be implemented by the storage 11 or the memory 12.

The control unit 230 can be implemented by the processor 13 loading the program stored in the storage 11 into the memory 12 and executing the program.

The communication I/F unit 250 can be implemented by the communication I/F 16.

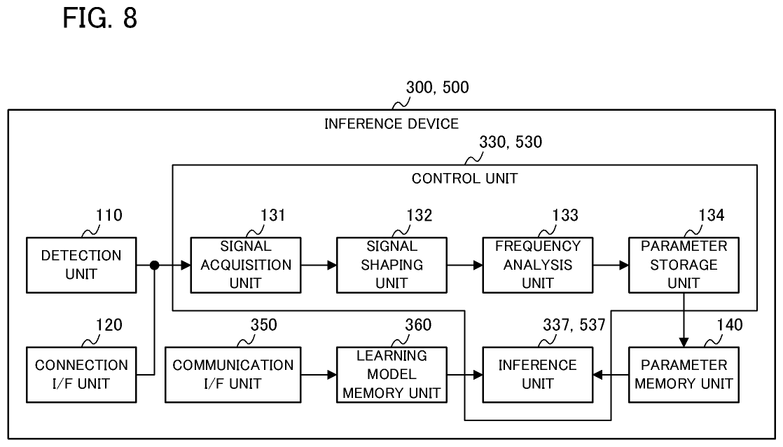

FIG. 8 is a block diagram schematically illustrating a configuration of the inference device 300 according to the second embodiment.

The inference device 300 includes a detection unit 110, a connection I/F unit 120, a control unit 330, a parameter memory unit 140, a communication I/F unit 350, and a learning-model memory unit 360.

The detection unit 110, the connection I/F unit 120, and the parameter memory unit 140 of the inference device 300 in the second embodiment are similar to the detection unit 110, the connection I/F unit 120, and the parameter memory unit 140 of the signal processing device 100 in the first embodiment.

The communication I/F unit 350 performs communication via the network 20.

The communication I/F unit 350 receives a learning model from the learning device 200 via the network 20, for example.

The learning-model memory unit 360 stores the learning model received by the communication I/F unit 350.

The control unit 330 controls processing in the inference device 300.

The control unit 330 includes a signal acquisition unit 131, a signal shaping unit 132, a frequency analysis unit 133, a parameter storage unit 134, and an inference unit 337.

The signal acquisition unit 131, the signal shaping unit 132, the frequency analysis unit 133, and the parameter storage unit 134 of the control unit 330 in the second embodiment are similar to the signal acquisition unit 131, the signal shaping unit 132, the frequency analysis unit 133, and the parameter storage unit 134 of the control unit 130 in the first embodiment.

The inference unit 337 inputs a period parameter, a power parameter, and a waveform shape parameter for each predetermined period stored in the parameter memory unit 140, into the learning model stored in the learning-model memory unit 360, thereby inferring an emotion corresponding to the parameters.

The parameters used by the inference unit 337 may correspond to the parameters used by the learning device 200 for training.

The inference device 300 described above can be implemented by the computer 10 illustrated in FIG. 3.

The parameter memory unit 140 and the learning-model memory unit 360 can be implemented by the storage 11 or the memory 12, for example.

The control unit 330 can be implemented by the processor 13 loading the program stored in the storage 11 into the memory 12 and executing the program.

The communication I/F unit 350 can be implemented by the communication I/F 16.

For example, the emotion recognition system 1 according to the second embodiment may be applied when classifying emotions, such as comfort and discomfort, or wakefulness and non-wakefulness, using a dataset of pulse wave signals labeled with these emotional states, such as comfort and discomfort, or wakefulness and non-wakefulness.

It is known that a person's internal state, that is, emotion, is reflected in a pulse waveform shape (waveform shape), which represents variations within a single waveform; it is also possible to perform training of a CNN (Convolutional Neural Network), by using only parameters of the pulse waveform shape (waveform shape) for multiple pulse beats. This makes it possible to separate the power from the pulse waveform shape, thereby isolating components related to the overall signal amplitude. Consequently, components of the pulse waveform shape derived from vascular biomechanics can be appropriately learned in the time-frequency domain, enabling accurate classification.

### Third Embodiment

In the second embodiment described above, classification is performed based on parameters by machine learning, however, waveform prediction may be performed by applying regression.

As illustrated in FIG. 6, a signal prediction system 2 as an information processing system according to a third embodiment includes a learning device 400 as an information processing device, and an inference device 500 as an information processing device.

Here, the learning device 400 and the inference device 500 are capable of transmitting and receiving data via the network 20 such as the Internet.

The learning device 400 performs processing during a learning phase, which is a phase for training a learning model.

The inference device 500 acquires a trained learning model from the learning device 400, and performs processing during an inference phase, which is a phase for predicting a future time-series signal from a time-series signal by using the trained learning model.

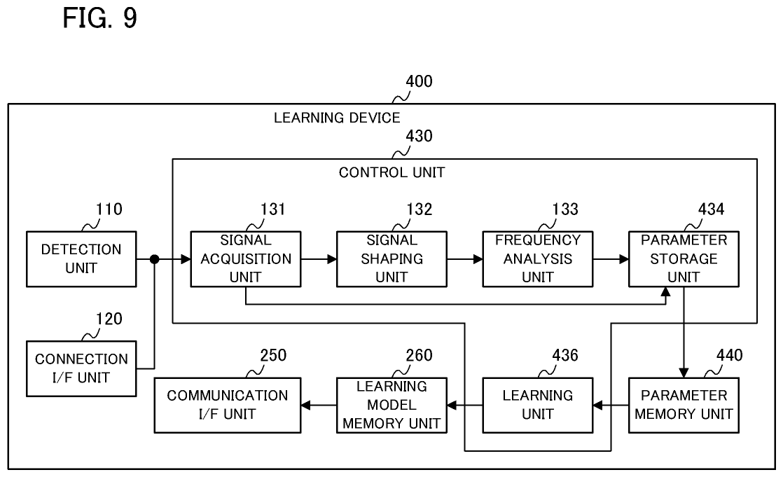

FIG. 9 is a block diagram schematically illustrating a configuration of the learning device 400 in the third embodiment.

The learning device 400 includes a detection unit 110, a connection I/F unit 120, a control unit 430, a parameter memory unit 440, a communication I/F unit 250, and a learning-model memory unit 260.

The detection unit 110 and the communication I/F unit 120 of the learning device 400 in the third embodiment are similar to the detection unit 110 and the communication I/F unit 120 of the signal processing device 100 in the first embodiment.

The communication I/F unit 250 and the learning-model memory unit 260 of the learning device 400 in the third embodiment are similar to the communication I/F unit 250 and the learning-model memory unit 260 of the learning device 200 in the second embodiment.

The control unit 430 controls processing in the learning device 400.

The control unit 430 includes a signal acquisition unit 131, a signal shaping unit 132, a frequency analysis unit 133, a parameter storage unit 434, and a learning unit 436.

The signal acquisition unit 131, the signal shaping unit 132, and the frequency analysis unit 133 of the control unit 430 in the third embodiment are similar to the signal acquisition unit 131, the signal shaping unit 132, and the frequency analysis unit 133 of the control unit 130 in the first embodiment.

However, the signal acquisition unit 131 also provides an acquired time-series signal to the parameter storage unit 434.

The parameter storage unit 434 stores a period parameter, a power parameter, and a waveform shape parameter from the frequency analysis unit 133, together with the time-series signal, in the parameter memory unit 440.

The parameter memory unit 440 stores the period parameter, the power parameter, and the waveform shape parameter, together with the time-series signal.

The learning unit 436 trains a learning model for predicting a future time-series signal by using the period parameter, the power parameter, and the waveform-shape parameter for each predetermined period stored in the parameter memory unit 440.

For example, the learning unit 436 generates a learning model for inferring the future time-series signal by using, as a training example, at least the power parameter corresponding to the time-series signal at a first time for learning, as a correct answer, the time-series signal at a second time, which is later than the first time. The power parameter, the period parameter, and the waveform shape parameter used for training are also referred to as a training power parameter, a training period parameter, and a training waveform shape parameter, respectively.

As a machine learning method used by the learning unit 436, a neural network can be exemplarily selected, although other machine learning methods may also be employed. As the parameter used for training, at least one of the period parameter, the power parameter, and the waveform shape parameter may be used. In this example, it is assumed that at least the power parameter is used.

The learning model trained as described above is stored in the learning-model memory unit 260 and transmitted to the inference device 500 by the communication I/F unit 250.

The learning device 400 described above can be implemented by the computer 10 illustrated in FIG. 3.

The parameter memory unit 440 and the learning-model memory unit 260 can be implemented by the storage 11 or the memory 12, for example.

The control unit 430 can be implemented by the processor 13 loading a program stored in the storage 11 into the memory 12 and executing the program.

The communication I/F unit 250 can be implemented by the communication I/F 16.

As illustrated in FIG. 8, the inference device 500 in the third embodiment includes a detection unit 110, a connection I/F unit 120, a control unit 530, a parameter memory unit 140, a communication I/F unit 350, and a learning-model memory unit 360.

The detection unit 110, the connection I/F unit 120, and the parameter memory unit 140 of the inference device 500 in the third embodiment are similar to the detection unit 110, the connection I/F unit 120, and the parameter memory unit 140 of the signal processing device 100 in the first embodiment.

The communication I/F unit 350 and the learning-model memory unit 360 of the inference device 500 in the third embodiment are similar to the communication I/F unit 350 and the learning-model memory unit 360 of the inference device 300 in the second embodiment.

The control unit 530 controls processing in the inference device 500.

The control unit 530 includes a signal acquisition unit 131, a signal shaping unit 132, a frequency analysis unit 133, a parameter storage unit 134, and an inference unit 537.

The signal acquisition unit 131, the signal shaping unit 132, the frequency analysis unit 133, and the parameter storage unit 134 of the control unit 530 in the third embodiment are similar to the signal acquisition unit 131, the signal shaping unit 132, the frequency analysis unit 133, and the parameter storage unit 134 of the control unit 130 in the first embodiment.

The inference unit 537 inputs the period parameter, the power parameter, and the waveform shape parameter for each predetermined period stored in the parameter memory unit 140, into a learning model stored in the learning-model memory unit 360, thereby inferring a future time-series signal from the parameters.

The parameter used by the inference unit 537 may correspond to the parameters used by the learning device 400 during learning.

The inference device 500 described above can be implemented by the computer 10 illustrated in FIG. 3.

The parameter memory unit 140 and the learning-model memory unit 360 can be implemented by the storage 11 or the memory 12, for example.

The control unit 530 can be implemented by the processor 13 loading a program stored in the storage 11 into the memory 12 and executing the program.

The communication I/F unit 350 can be implemented by the communication I/F 16.

For example, the signal processing described in the third embodiment may be applied for predicting future daily average temperatures in Tokyo based on approximately ten years of data recording changes in daily average temperatures in Tokyo. In this case, the waveform shape represents the way of seasonal transitions in Tokyo, the power represents changes in the magnitude of temperature fluctuations throughout the year, and the period represents the cycle of the seasonal transitions. Here, since the period does not change from one year, the period parameter is not required for training. Accordingly, by using the two parameters, i.e., the waveform shape parameter and the power parameter, training by using LSTM (Long Short-Term Memory) is performed separately for each parameter, thereby enabling more appropriate inference compared to conventional parameterization and learning methods.

In this case, for example, it is sufficient to train the learning model so as to predict the time-series signal for the subsequent year based on parameters from the past ten years.

### Fourth Embodiment

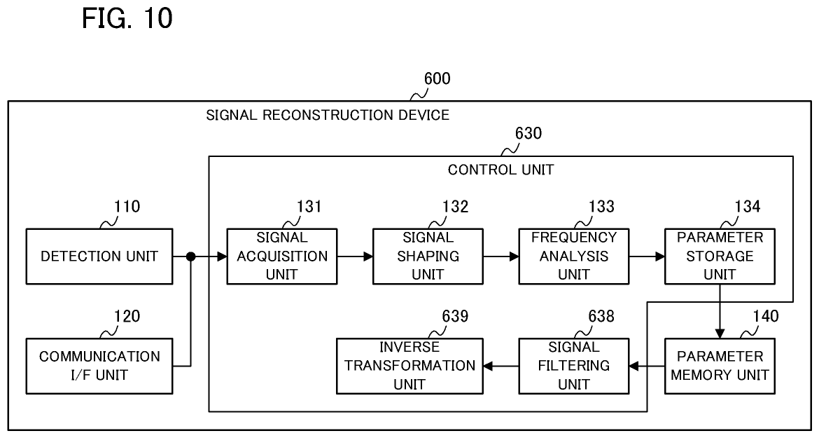

FIG. 10 is a block diagram schematically illustrating a configuration of a signal reconstruction device 600 that is an information processing device according to a fourth embodiment.

The signal reconstruction device 600 includes a detection unit 110, a connection I/F unit 120, a control unit 630, and a parameter memory unit 140.

FIG. 10 illustrates that the signal reconstruction device 600 includes both the detection unit 110 and the connection I/F unit 120; however, the signal reconstruction device 600 may include at least one of them.

The detection unit 110, the connection I/F unit 120, and the parameter memory unit 140 of the signal reconstruction device 600 according to the fourth embodiment are similar to the detection unit 110, the connection I/F unit 120, and the parameter memory unit 140 of the signal processing device 100 according to the first embodiment.

The control unit 630 controls processing in the signal reconstruction device 600.

The control unit 630 includes a signal acquisition unit 131, a signal shaping unit 132, a frequency analysis unit 133, a parameter storage unit 134, a signal filtering unit 638, and an inverse transformation unit 639.

The signal acquisition unit 131, the signal shaping unit 132, the frequency analysis unit 133, and the parameter storage unit 134 of the control unit 630 in the fourth embodiment are similar to the signal acquisition unit 131, the signal shaping unit 132, the frequency analysis unit 133, and the parameter storage unit 134 of the control unit 130 in the first embodiment.

The signal filtering unit 638 performs filtering on at least one of the power parameter, the period parameter, and the waveform shape parameter.

For example, the signal filtering unit 638 performs processing to remove an unnecessary component from the period parameter, the power parameter, and the waveform shape parameter stored in the parameter memory unit 140, or to replace the unnecessary component therein with a constant.

Specifically, the signal filtering unit 638 replaces the period parameter with a constant for eliminating periodic variations in a time-series signal.

Further, the signal filtering unit 638 replaces the power parameter with a constant for eliminating temporal variations in the amplitude of a time-series signal.

Furthermore, the signal filtering unit 638 replaces the harmonic components of the waveform shape parameter with zero for eliminating harmonic components of a time-series signal and representing the signal as a simple sine wave.

The parameters filtered as described above are provided to the inverse transformation unit 639.

The inverse transformation unit 639 converts, into a time-series signal, the power parameter, the period parameter, and the waveform shape parameter which are filtered by the signal filtering unit 638.

For example, the inverse transformation unit 639 performs an inverse complex Fourier series expansion for each waveform by using the parameters filtered by the signal filtering unit 638, thereby calculating a reconstruction signal.

For example, in a pulse wave signal as a time-series signal acquired by a pulse wave Doppler sensor, since the distance to a signal provider may affect the amplitude of the pulse wave signal, amplitude information of each waveform may be regarded as noise in some cases. In these cases, by replacing pulse wave power with a constant that does not vary for each waveform with the use of parameters of a pulse period and a pulse wave shape, and then by performing an inverse transformation, it is possible to output a reconstruction signal from which temporal variations in signal amplitude have been removed.

The signal reconstruction device 600 described above can be implemented by the computer 10 illustrated in FIG. 3.

For example, the control unit 630 can be implemented by the processor 13 loading a program stored in the storage 11 into the memory 12 and executing the program.

What is claimed is:

1. An information processing device comprising:
a processor to execute a program; and
a memory to store the program which, when executed by the processor, performs processes of,
acquiring a time-series signal indicating a plurality of waveforms;
identifying a single waveform from the time-series signal; and
calculating a power parameter that is a parameter indicating power of the single waveform, a period parameter that is a parameter indicating a period of the single waveform, and a waveform shape parameter that is a spectrum calculated by normalizing a frequency spectrum by using a square root of sum of squares, the period parameter being calculated by a square root of sum of squares of the frequency spectrum.

2. The information processing device according to claim 1, wherein the processor is configured to calculate a period of the single waveform or a fundamental wave frequency of the single waveform, and calculate the power parameter, the period parameter, and the waveform shape parameter by performing frequency analysis processing.

3. The information processing device according to claim 2, wherein the frequency analysis processing includes a complex Fourier series expansion.

4. The information processing device according to claim 3, wherein the processor is configured to calculate the frequency spectrum by the complex Fourier series expansion, set, as the power parameter, the square root of sum of squares of the frequency spectrum, and set, as the waveform shape parameter, a spectrum calculated by normalizing the frequency spectrum by using a square root of sum of squares.

5. The information processing device according to claim 1, wherein
the time-series signal is a signal indicating a pulse wave detected from a person, and
the processor is configured to generate a learning model for inferring an emotion of the person, by using the power parameter as a training example for learning, as a correct answer, an emotion of the person at a time when the single waveform is acquired.

6. The information processing device according to claim 1, wherein
the time-series signal is a signal indicating a pulse wave detected from a person, and
the processor is configured to infer an emotion of the person by inputting the power parameter into a learning model, the learning model being generated by using, as a training example, a training power parameter that is a parameter indicating power of a training single waveform identified from a signal indicating a pulse wave detected from a learning subject for learning, as a correct answer, an emotion of the learning subject at a time when the training single waveform is acquired.

7. The information processing device according to claim 1, wherein
the processor is configured to generate a learning model for inferring a future time-series signal, by using, as a training example, the power parameter corresponding to the time-series signal at a first time for learning, as a correct answer, the time-series signal at a second time, which is later than the first time.

8. The information processing device according to claim 1, wherein
the processor is configured to infer a future time-series signal by inputting the power parameter into a learning model, the learning model being generated by using, as a training example, a training power parameter indicating power of a single waveform identified from a time-series signal indicating a plurality of waveforms at a first time for learning, as a correct answer, a time-series signal indicating a waveform at a second time, which is later than the first time.

9. The information processing device according to claim 1, wherein
the processor is configured to perform filtering processing on at least one of the power parameter, the period parameter, and the waveform shape parameter; and to convert the power parameter, the period parameter, and the waveform shape parameter after the filtering processing, into a time-series signal.

10. A non-transitory computer-readable storage medium storing a program that causes a computer to execute processing comprising:
acquiring a time-series signal indicating a plurality of waveforms;
identifying a single waveform from the time-series signal; and
calculating a power parameter that is a parameter indicating power of the single waveform, a period parameter that is a parameter indicating a period of the single waveform, and a waveform shape parameter that is a spectrum calculated by normalizing a frequency spectrum by using a square root of sum of squares, the period parameter being calculated by a square root of sum of squares of the frequency spectrum.

11. An information processing method comprising:
acquiring a time-series signal indicating a plurality of waveforms;
identifying a single waveform from the time-series signal; and
calculating a power parameter that is a parameter indicating power of the single waveform, a period parameter that is a parameter indicating a period of the single waveform, and a waveform shape parameter that is a spectrum calculated by normalizing a frequency spectrum by using a square root of sum of squares, the period parameter being calculated by a square root of sum of squares of the frequency spectrum.
